(19) United States
(12) Patent Application Publication
(10) Pub. No.: US 2023/0181668 A1
(43) Pub. Date: Jun. 15, 2023

(54) Dehydrated Aloe Vera Impregnated Medicated Gauze

(71) Applicant: Lorena Bernal, Anaheim, CA (US)

(72) Inventor: Lorena Bernal, Anaheim, CA (US)

(21) Appl. No.: 18/053,938

(22) Filed: Nov. 9, 2022

Related U.S. Application Data
(60) Provisional application No. 63/289,234, filed on Dec. 14, 2021.

(51) Int. Cl. A61K 36/886; A61L 15/40

(52) U.S. Cl. CPC A61K 36/886; A61L 15/40

(57) ABSTRACT

The present invention relates to a medicated gauze impregnated with a dehydrated aloe vera extract. The medicated gauze is designed to treat deep wounds, topical burns and dry skin. The medicated gauze is produced by soaking the gauze in an aloe vera extract, drying the gauze in a sterile dehydrator, and then rolling the dried gauze into packages. When the medicated gauze is applied to a wound, the dehydrated aloe vera extract is hydrated by the patient's body moisture to create an aloe vera gel environment conducive to preventing infection and promoting wound healing while avoiding damage to surrounding skin.

### CROSS-REFERENCE TO RELATED APPLICATION

The present application claims priority to, and the benefit of, U.S. Provisional Application No. 63/289,234, which was filed on Dec. 14, 2021, and is incorporated herein by reference in its entirety.

### FIELD OF THE INVENTION

The present invention relates generally to the field of medicated gauzes and bandages. More specifically, the invention relates to an improved antimicrobial gauze. The gauze is impregnated with a dehydrated aloe vera extract. The dehydrated aloe vera extract in the gauze is hydrated by a patient's natural body moisture when the gauze is applied to a patient's wound. Accordingly, this disclosure makes specific reference thereto the present invention. Nonetheless, it is to be appreciated that aspects of the present invention are also equally applicable to other like applications, devices, and methods of manufacture.

### BACKGROUND OF THE INVENTION

Wound healing can be a long, painful process. Open wounds increase the chance of infection. Gauze, held in place by bandages or tape, has been used to relieve pain, prevent infection, and promote healing of wounds. Gauze is used because it easily conforms to the wound's shape. However, gauze may adhere to a wound such that when the gauze is removed from a wound it may also remove portions of new skin or undamaged skin surrounding the wound; thus, reversing or delaying the healing process.

To prevent adherence of gauze to a healing wound, the gauze may be moistened with normal saline (NS), or ointment. Medical grade antimicrobial gels, when applied on gauze, can prevent adherence of gauze to a wound and also prevent infection. However, the medical grade gels are synthetically produced; therefore, they are expensive and may cause adverse reactions for some patients. In addition, the gels, ointments, and other topicals agents used on gauze can cause further skin breakdown of the affected area of the wound and healthy skin around the wound when oversaturated with the topical agent.

Therefore, there exists a long-felt need in the art for a hypoallergenic, medicated gauze that relieves pain, prevents infection, promotes wound healing, and does not damage the wound or healthy areas around the wound or cause other adverse reactions. Furthermore, there is a long-felt need in the art for a method of producing a medicated gauze that will allow for an affordable product in high volume. Finally, there is a long-felt need in the art for a method of using a medicated gauze to treat deep wounds, topical burns, and dry skin.

The subject matter disclosed and claimed herein addresses these long-felt needs. In one embodiment, the present invention comprises a medicated gauze. The medicated gauze includes an antimicrobial layer, an absorbent layer wherein the antimicrobial layer and the absorbent layer are impregnated with a dehydrated aloe vera extract. In another embodiment, the present invention provides for a method of producing a medicated gauze, an aloe vera extract is impregnated into the gauze by soaking the gauze in the aloe vera extract and then drying the gauze in a sterile dehydrator. In yet another embodiment, the present invention provides for a method to treat deep wounds, topical burns, and dry skin. When applied on a wound, the dehydrated aloe vera in the medicated gauze is hydrated by the patient's body moisture naturally present in various wound stages.

In this manner, the medicated gauze of the present invention accomplishes all the forementioned objectives. The medicated gauze of the present invention is a hypoallergenic, medicated gauze that relieves pain, prevents infection, promotes wound healing, does not damage the wound or healthy areas around the wound, or cause other adverse reactions for the patient. Further, the medicated gauze allows for a method of production such that it is affordable and producible in high volume. Finally, the medicated gauze allows for a method to treat wounds, topical burns, and dry skin.

### SUMMARY OF THE INVENTION

The following presents a simplified summary to provide a basic understanding of some aspects of the disclosed innovation. This summary is not an extensive overview, and it is not intended to identify any key or critical elements or to delineate the scope thereof. Its sole purpose is to present some general concepts in a simplified form as a prelude to the more detailed description that is presented later.

The subject matter disclosed and claimed herein, in one embodiment thereof, comprises a medicated gauze. The medicated gauze is comprised of an antimicrobial layer and an absorbent layer; wherein, the antimicrobial layer and the absorbent layer are impregnated with a dehydrated aloe vera extract. The antimicrobial layer may be formed from antimicrobial fibers. The absorbent layer may be formed from absorbent fibers. The aloe vera extract is comprised of aloe vera gel. The aloe vera gel is extracted naturally from aloe vera barbadensis or aloe vera barbadensis Miller.

The present invention may also include a process/method of producing a medicated gauze impregnated with a dehydrated aloe vera extract as described. The steps in the method include soaking a gauze in an aloe vera extract for a first predetermined period of time, placing the soaked gauze in a sterile dehydrator device for a second predetermined period of time for drying the gauze, and packaging the dried gauze as a roll in a sterile packaging.

Additionally, the present invention may include a method for the therapeutic treatment of deep wounds, topical burns and dry skin. The method includes providing a medicated gauze impregnated with dehydrated aloe vera extract, applying the medicated gauze to an open wound, rehydrating the dehydrated aloe vera extract with the patient's body moisture, and creating an antimicrobial gel environment over the wound to promote the healing process without over-saturating the wound or the healthy skin surrounding the wound with a topical agent. The antimicrobial gel environment produced by the medicated gauze increases the amount of collagen and collagen cross-linking; thereby, promoting wound healing.

Numerous benefits and advantages of this invention will become apparent to those skilled in the art to which it pertains upon reading and understanding of the following detailed specification.

To the accomplishment of the foregoing and related ends, certain illustrative aspects of the disclosed innovation are described herein in connection with the following description and the annexed drawings. These aspects are indicative, however, of but a few of the various ways in which the principles disclosed herein can be employed and are intended to include all such aspects and their equivalents. Other advantages and novel features will become apparent from the following detailed description when considered in conjunction with the drawings.

### BRIEF DESCRIPTION OF THE DRAWINGS

The description refers to provided drawings in which similar reference characters refer to similar parts throughout the different views, and in which:

FIG. 1A illustrates a perspective view of one potential embodiment of the medicated gauze impregnated with dehydrated aloe vera extract in accordance with the disclosed architecture;

FIG. 1B illustrates the composition of the aloe vera extract of one potential embodiment of the present invention in accordance with the disclosed architecture;

FIG. 2 illustrates a flow diagram showing steps in producing one potential embodiment of the medicated gauze of the present invention in accordance with the disclosed architecture;

FIG. 3 illustrates standalone rolls of one potential embodiment of the medicated gauze of the present invention in accordance with the disclosed architecture;

FIG. 4 illustrates one potential embodiment of the medicated gauze of the present invention incorporated into a bandage in accordance with the disclosed architecture; and

FIG. 5 illustrates a flow diagram showing exemplary steps performed in treating a wound with one potential embodiment of the medicated gauze of the present invention in accordance with the disclosed architecture.

### DETAILED DESCRIPTION OF THE PRESENT INVENTION

The innovation is now described with reference to the drawings, wherein like reference numerals are used to refer to like elements throughout. In the following description, for purposes of explanation, numerous specific details are set forth to provide a thorough understanding thereof. It may be evident, however, that the innovation can be practiced without these specific details. In other instances, well-known structures and devices are shown in block diagram form to facilitate a description thereof. Various embodiments are discussed hereinafter. It should be noted that the figures are described only to facilitate the description of the embodiments. They are not intended as an exhaustive description of the invention and do not limit the scope of the invention. Additionally, an illustrated embodiment need not have all the aspects or advantages shown. Thus, in other embodiments, any of the features described herein from different embodiments may be combined.

As noted above, there exists a long-felt need in the art for a hypoallergenic, medicated gauze that relieves pain, prevents infection, promotes wound healing, and does not damage the wound or healthy areas around the wound, or cause other adverse reactions. Furthermore, there is a long-felt need in the art for a method of producing a medicated gauze that will allow for an affordable product in high volume. Finally, there is a long-felt need in the art for a method of using a medicated gauze to treat deep wounds, topical burns, and dry skin.

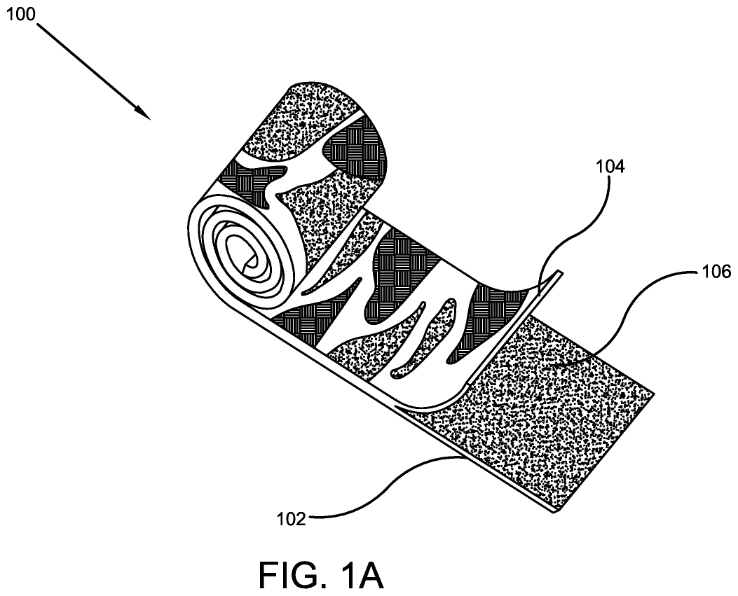

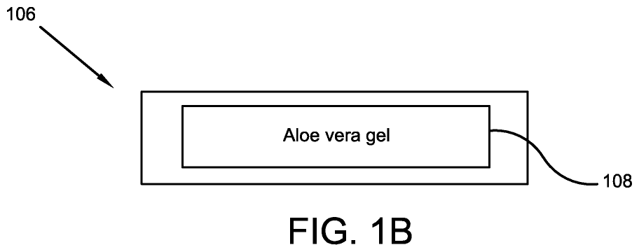

Referring initially to the drawings, FIG. 1A illustrates a perspective view of one exemplary embodiment of the invention, a medicated gauze impregnated with a dehydrated aloe vera extract, in accordance with the disclosed architecture. The medicated gauze 100 of the present invention is designed as an affordable and hypoallergenic medicated gauze or bandage that combines a conventional gauze with the unique healing properties of the aloe vera plant as a topical applicant. The medicated gauze 100 works to promote quick and easy healing of dry, damaged skin, and deep wounds. It should be noted that the term “medicated” in the present description is meant that the gauze, bandage, or patch is comprised of the composition hereto described in accordance with the present invention, e.g., a gauze impregnated with a dehydrated aloe vera extract.

More specifically, the medicated gauze 100 includes an antimicrobial layer 102 made from antimicrobial fibers and an absorption layer 104 made from absorbent fibers. The gauze 100 has a dehydrated aloe vera extract 106 as an active ingredient. The antimicrobial layer and the absorbent layer are impregnated with the dehydrated aloe vera extract 106. The antimicrobial layer 102 contacts the wound and surrounding skin when the gauze 100 is applied to a wound. The body moisture present in the wound hydrates the aloe vera extract to create an aloe vera gel environment only in the area of the wound; thus, preventing surrounding skin from becoming oversaturated and damaged by a topical agent. The gauze 100 is preferably hypoallergenic, paraben-free, sulfate-free, triclosan-free, and vegan.

Referring to FIG. 1B, the composition of the aloe vera extract 106 of the medicated gauze 100 is illustrated. The aloe vera extract 106 is comprised of aloe vera gel 108. The relative composition of these components is approximately 100% (by weight) aloe vera gel 108.

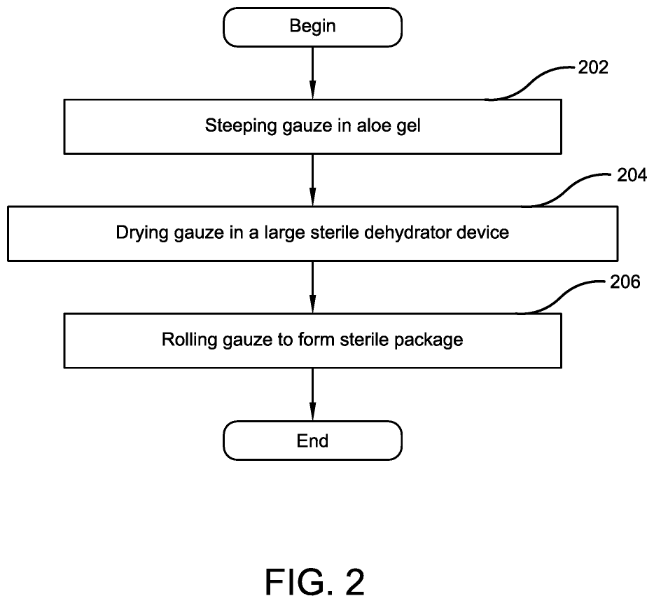

In another exemplary embodiment of the present invention, a method for producing a medicated gauze impregnated with a dehydrated aloe vera extract is described. FIG. 2 illustrates a flow diagram showing steps in the production of the medicated gauze 100 of the present invention in accordance with the disclosed architecture. Initially, a gauze is soaked for approximately 1-2 hours in an aloe vera extract, wherein said aloe vera extract is derived from an aloe vera plant such as but not limited to aloe vera barbadensis or aloe vera barbadensis Miller (Step 202). Then, the gauze is placed in a sterile dehydrator providing uniform air circulation for approximately 24 to 48 hours; thereby, causing the gauze to be impregnated with a dehydrated aloe vera extract (Step 204). Thereafter, the gauze is rolled to form a sterile package available in standard rolls of various lengths (Step 206). The medicated gauze 100, produced in this manner can be stored for prolonged periods of time, and can be cut into various lengths and widths for use on differently sized wounds.

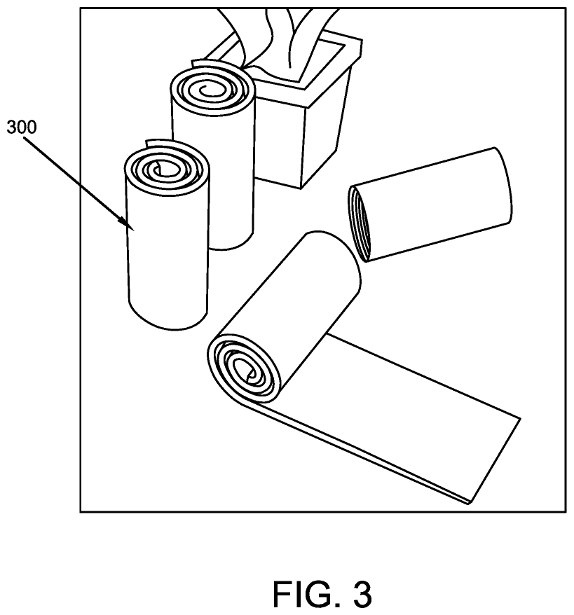

FIG. 3 illustrates standalone rolls of the medicated gauze 100 of the present invention in accordance with the disclosed architecture. The gauze 100 is available in standalone rolls 400 of various sizes such as, but not limited to: 4 cm×50 cm, 3 cm×40 cm and 6 cm×100 cm for example. Further, the gauze 100 may come in paraffin white color or any color that may or may not match the skin tone of a user. The rolls 300 are contained in sterile packaging and can be cut into various lengths and widths for use on differently size wounds.

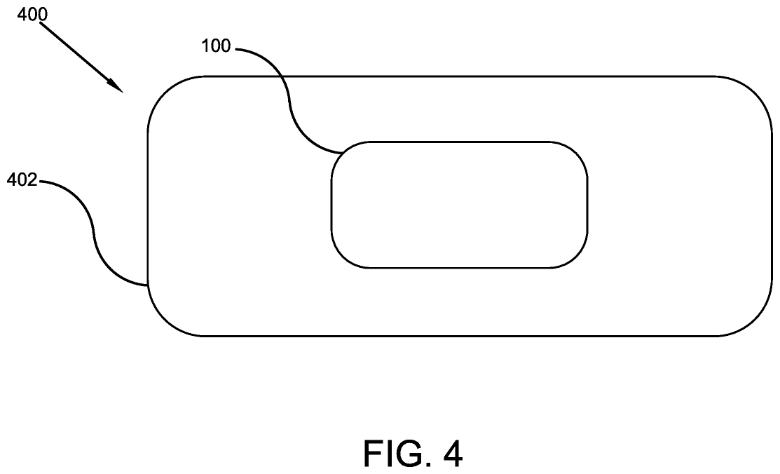

FIG. 4 illustrates how the mediated gauze 100 can be incorporated into a bandage. The bandage 400 having an adhesive area 402 which adheres to a patient's skin and a non-adhesive area composed of the medicated gauze 100. The bandages can be made in various sizes and shapes.

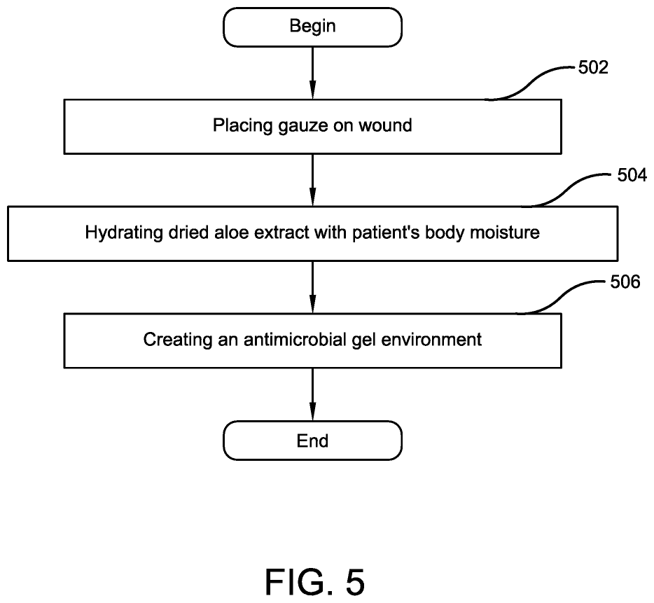

FIG. 5 illustrates a flow diagram showing exemplary steps performed in treating a wound with the dehydrated aloe vera impregnated medicated gauze in accordance with the disclosed architecture. Initially, the gauze 100 is placed loosely over or packed into the wound (Step 502). Then, the dehydrated aloe vera extract is hydrated by the patient's body moisture, naturally present during various wound stages (Step 504). The hydrated aloe vera extract creates an antimicrobial gel environment which quickens the healing process of the wound without over-saturating the wound and surrounding skin (Step 506). The medicated gauze 100 can be used to treat deep wounds as well as for topical burns and dry skin to promote quick and natural healing.

Certain terms are used throughout the following description and claims to refer to particular features or components. As one skilled in the art will appreciate, different persons may refer to the same feature or component by different names. This document does not intend to distinguish between components or features that differ in name but not structure or function. As used herein “medicated gauze”, “medicated gauze”, “gauze”, “bandage” and “dehydrated aloe vera impregnated medicated gauze” are interchangeable and refer to the dehydrated aloe vera impregnated medicated gauze 100 of the present invention.

Notwithstanding the forgoing, the dehydrated aloe vera impregnated medicated gauze 100 of the present invention can be of any suitable size and configuration as is known in the art without affecting the overall concept of the invention, provided it accomplishes the above-stated objectives. One of ordinary skill in the art will appreciate that the dehydrated aloe vera impregnated medicated gauze 100 as shown in the FIGS. are for illustrative purposes only, and that many other sizes and shapes of the dehydrated aloe vera impregnated medicated gauze 100 are well within the scope of the present disclosure. Although the dimensions of the dehydrated aloe vera impregnated medicated gauze 100 are important design parameters for user convenience, the dehydrated aloe vera impregnated medicated gauze 100 may be of any size that ensures optimal performance during use and/or that suits the user's needs and/or preferences.

Various modifications and additions can be made to the exemplary embodiments discussed without departing from the scope of the present invention. While the embodiments described above refer to particular features, the scope of this invention also includes embodiments having different combinations of features and embodiments that do not include all the described features. Accordingly, the scope of the present invention is intended to embrace all such alternatives, modifications, and variations as fall within the scope of the claims, together with all equivalents thereof.

What has been described above includes examples of the claimed subject matter. It is, of course, not possible to describe every conceivable combination of components or methodologies for purposes of describing the claimed subject matter, but one of ordinary skill in the art may recognize that many further combinations and permutations of the claimed subject matter are possible. Accordingly, the claimed subject matter is intended to embrace all such alterations, modifications and variations that fall within the spirit and scope of the appended claims. Furthermore, to the extent that the term “includes” is used in either the detailed description or the claims, such term is intended to be inclusive in a manner similar to the term “comprising” as “comprising” is interpreted when employed as a transitional word in a claim.

What is claimed is:

1. A medicated gauze comprising:
a dehydrated aloe vera extract;
an antimicrobial layer; and
an absorption layer.

2. The medicated gauze of claim 1, wherein the antimicrobial layer is impregnated with the dehydrated aloe vera extract.

3. The medicated gauze of claim 1, wherein the absorption layer is impregnated with the dehydrated aloe vera extract.

4. The medicated gauze of claim 1, wherein the dehydrated aloe vera extract is comprised of an aloe vera gel.

5. The medicated gauze of claim 4, wherein the dehydrated aloe vera extract is comprised of 100% aloe vera gel.

6. The medicated gauze of claim 1, wherein the aloe vera extract is derived from an aloe vera barbadensis plant or an aloe vera barbadensis Miller plant.

7. A process for producing a medicated gauze comprising the steps of:
soaking a gauze in an aloe vera extract;
drying the gauze in a sterile dehydrator; and
rolling the gauze to form a package of medicated gauze.

8. The process for producing a medicated gauze of claim 7, wherein the soaking of the gauze in the aloe vera extract occurs for at least one hour.

9. The process for producing a medicated gauze of claim 7, wherein the gauze is comprised of an antimicrobial layer and an absorption layer.

10. The process for producing a medicated gauze of claim 7, wherein the aloe vera extract is comprised of an aloe vera gel.

11. The process for producing a medicated gauze of claim 10, wherein the aloe vera extract is comprised of 100% aloe vera gel.

12. The process for producing a medicated gauze of claim 7, wherein the aloe vera extract is derived from an aloe vera barbadensis plant or an aloe vera barbadensis Miller plant.

13. The process for producing a medicated gauze of claim 7, wherein the gauze is dried in the sterile dehydrator for a duration of time ranging from 24 hours to 48 hours.

14. A process for treating a wound comprising the steps of:
providing a medicated gauze; and
laying the medicated gauze over the wound or packing the medicated gauze into the wound, wherein a moisture from the wound hydrates the medicated gauze.

15. The process for treating a wound of claim 14, wherein the medicated gauze is comprised of an antimicrobial layer, an absorption layer and a dehydrated aloe vera extract.

16. The process for treating a wound of claim 15, wherein the antimicrobial layer is impregnated with the dehydrated aloe vera extract.

17. The process for treating a wound of claim 15, wherein the absorption layer is impregnated with the dehydrated aloe vera extract.

18. The process for treating a wound of claim 15, wherein the dehydrated aloe vera extract is comprised of an aloe vera gel.

19. The process for treating a wound of claim 15, wherein the dehydrated aloe vera extract is comprised of 100% aloe vera gel.

20. The process for treating a wound of claim 15, wherein the dehydrated aloe vera extract is derived from an aloe vera barbadensis plant or an aloe vera barbadensis Miller plant.
